SEQUENCE LISTING (19 sequences)

SEQ ID NO: 1 - DNA, 1175 bp, Homo sapiens
caacacctgcaaggctgtgcgggctcctggccgcaccctatcggccgggaagaacaagcgagggagttcctactccaggaccgtggggttctcccgcggctctgacagtccccaggagggagccccaaactcccggtgccagctgcagccgcctcgctttccgagaagctttcggaagcttttcgaagtcggagaagcaactgccccggggctcacctttgcgctgaagaggtgccgggagtctcggggaaaaggcaccgctgtacagaaaggaaacattgtaggggggttagccgaccggcaacatcgcgacgaacccggcgaccccacgccccaacaccctagcccgggaattcccccttcttcagacagatacccaccccgcaccctcttttaatccggggcaggaggtatccgtccctggggcacccgcccacctcccaccacggcgaggggcgctgcacagggggaaagggagggcttgcaccccaaccaagctgctttgtgcaacgctgtcacccgaaatccgccacggcccccacgcacgcccccggagtggggagcctcccacctctgagcgccagcgaggcggtgcgcgccgccctggactgcgggcgcctctccggagccgcgcgccgcgcgcacaggacggctgcagccgcgccgcgagccccagcctcttctcggccgccgccgccgccgccgcttccgcccctgtcacggcgtgacccggggtgggaggagccgacaggactagggatgggtagggtccccgccttcgctttagccgcagcgctcgctccgccctgcccatttccgccgctgcgcaaaccccggcgcttggggaagtgggtggagtctgtgcaacgaggtacggggcgggagtggagacggggcgccgggagagggggcgggaactgaggcggggcgcgggaaagaggcggggcgcgggaaagaggcggggcgcgggcggaggcggggcgcggagaagtggcggaggtggaagcggaggcgtacccgcccctggggacgtcattggtggcggaggcaatggccggcaaccagctgtaagcgaggtaggctcactcgggcgcggagggtgcgggtgagaaagggaacgatttgctaggagtgtatgcgcccgtgctagggctcccakgrattcttacaagcgt

SEQ ID NO: 2 - DNA, 50 bp, Homo sapiens
gccccagcctcttctcggccgccgccgccgccgccgcttccgcccctgtc

SEQ ID NO: 3 - DNA, 47 bp, Homo sapiens
gccccagcctcttctcggccgccgccgccgccgcttccgcccctgtc

SEQ ID NO: 4 - DNA, 53 bp, Homo sapiens
gccccagcctcttctcggccgccgccgccgccgccgccgcttccgcccctgtc

SEQ ID NO: 5 - DNA, 18 bp, Homo sapiens
gccgccgccgccgccgcc

SEQ ID NO: 6 - DNA, 15 bp, Homo sapiens
gccgccgccgccgcc

SEQ ID NO: 7 - DNA, 21 bp, Homo sapiens
gccgccgccgccgccgccgcc

SEQ ID NO: 8 - DNA, 72 bp, Homo sapiens
agccccagcctcttctcggccgccgccgccgccgccgcttccgcccctgtcacggcgtgacccggggtggga

SEQ ID NO: 9 - DNA, 72 bp, Pan troglodytes; identical to SEQ ID NO: 8

SEQ ID NO: 10 - DNA, 72 bp, Erinaceus europeaus
agccgcagccccagttcggccgccgccgccgccgccgcttccgcccctgtcacggcgtgacccggggtggga

SEQ ID NO: 11 - DNA, 72 bp, Bos taurus
agccgcagcctcaactcggccgccgccgccgccgccgcttccgcccctgtcacgacgtgacccgtggtggga

SEQ ID NO: 12 - DNA, 63 bp, Rattus norvegicus
agctgcagcagctcggccgccgccgccgcttccgcccctatcacgccgtgacccggagtggga

SEQ ID NO: 13 - DNA, 63 bp, Oryctolagus cuniculus
agccgcagcccagctcggccgccgccaccgcccgcccctctggcggcgtgacccggagtggga

SEQ ID NO: 14 - DNA, 60 bp, Mus musculus
agctgcagcagctcggccgccgccgcttccgcccctatcacgccgtgacccggagtggga

SEQ ID NO: 15 - DNA, 72 bp, Monodelphis domestica
agccgcagcctgcgctcagccgccggcggcgccgctgcttccgcccctgtcacgccgtgacccggtggggga

SEQ ID NO: 16 - DNA, 65 bp, Otolemur garnettii
agtcatggcccctgcttggccgcagcggcctttgtgactcctggctctctcatgggccaatctgg

SEQ ID NO: 17 - DNA, 47 bp, Homo sapiens
cctcttctcggccgccgccgccgccgccgcttccgcccctgtcacgg

SEQ ID NO: 18 - DNA, 50 bp, Homo sapiens
cctcttctcggccgccgccgccgccgccgccgcttccgcccctgtcacgg

SEQ ID NO: 19 - DNA, 41 bp, Homo sapiens
cctcttctcggccgccgccgccgccgcttccgcccctgtca

(19) United States
(12) Patent Application Publication
(10) Pub. No.: US 2011/0300535 A1
(43) Pub. Date: Dec. 8, 2011

(54) METHOD OF IDENTIFYING INDIVIDUALS AT RISK OF THIOPURINE DRUG RESISTANCE AND INTOLERANCE

(75) Inventors: Rebecca Lee Roberts, Christchurch (NZ); Richard Blair Gearry, Christchurch (NZ); Murray Lindsay Barclay, Christchurch (NZ); Martin Alexander Kennedy, Christchurch (NZ)

(73) Assignee: University of Otago

(21) Appl. No.: 12/672,620

(22) PCT Filed: Aug. 7, 2008
(86) PCT No.: PCT/NZ2008/000197

§ 371 Date: Jun. 8, 2011

Related U.S. Application Data
(60) Provisional application No. 60/935,367, filed on Aug. 9, 2007.

(51) Int. Cl. C12Q 1/68; C07H 21/04

(52) U.S. Cl. 435/6.11; 536/24.31; 536/24.33

(57) ABSTRACT

The invention relates to methods and kits for identifying individuals at risk of thiopurine drug intolerance based on detecting the presence of mutations in the TPMT gene promoter associated with thiopurine drug resistance or intolerance.

### FIELD OF THE INVENTION

The present invention relates to methods and kits for identifying individuals at risk of thiopurine drug intolerance. These methods and kits are based on detecting the presence of mutations in the TPMT gene promoter associated with thiopurine drug resistance or intolerance.

### BACKGROUND TO THE INVENTION

Thiopurine drugs (predominantly azathioprine and 6-mercaptopurine) are used to treat a wide range of diseases. Amongst these are acute lymphoblastic leukemia, complications associated with solid organ transplantation, autoimmune diseases such as rheumatoid arthritis and inflammatory bowel disease (IBD) and dermatological conditions.

Thiopurine drugs are inactive and are metabolised in the body to the active metabolites 6-methylmercaptopurine ribonucleotides (6-MMPR) and 6-thioguanine nucleotide (6-TGN). 6-TGNs are beneficial, whereas 6-MMPR can be toxic. Unfortunately, up to 40% of individuals demonstrate drug resistance or intolerance to treatment using thiopurines. A proportion of individuals that are resistant to thiopurine treatment are unable to achieve therapeutic levels of 6-TGN, and instead accumulate 6-MMPR to hepatotoxic levels (>5700 pmol/8×10⁸ RBC).

Thiopurine S-methyltransferase (TPMT) is a cytoplasmic enzyme that catalyses the S-methylation of the thiopurine drugs. This enzyme is polymorphic with around 0.6% of Caucasians exhibiting complete deficiency, 11% intermediate activity and 89% normal TPMT activity in red blood cells (RBCs) (Wang et al, 2006). TPMT deficiency, which increases risk of myelotoxicity from these drugs, has been studied for over 25 years and is one of the few pharmacogenetic examples that has transitioned from research to the diagnostic setting. U.S. Pat. No. 5,856,095, for example shows genetic mutations in the TPMT gene that result in a decreased TPMT activity and relate directly with potentially fatal hematopoietic toxicity when patients are treated with a standard dose of thiopurine drugs and thus are linked directly with thiopurine intolerance or resistance. Unfortunately, not all patients who are thiopurine resistant or intolerant exhibit decreased TPMT activity. About 1-2% of Caucasians exhibit extremely high TPMT activity, which can result in thiopurine treatment resistance or hepatotoxicity. To date there is no satisfactory molecular explanation for this TPMT ultra-metaboliser (UM) phenotype.

An assay or method that provides a means of identifying individuals with extremely high TPMT activity who are at risk of thiopurine resistance or intolerance would be useful to practitioners attempting to establish such a risk in individuals in need of thiopurine therapy.

It is therefore an object of the present invention to provide methods and kits for identifying individuals with a TPMT UM phenotype who are at risk of thiopurine resistance or intolerance or to at least provide the public with a useful choice.

### SUMMARY OF THE INVENTION

The present inventors have surprisingly discovered previously unrecognised mutations present in the thiopurine S-methyltransferase (TPMT) gene that are associated with the UM phenotype and with individuals' response to thiopurine therapy. More particularly, mutations in the promoter region of TPMT have been identified as being associated with the UM phenotype and with a risk of drug resistance or intolerance to thiopurine therapy in individuals undergoing such therapy.

In this specification, positions are indicated with reference to SEQ ID NO: 1 unless the context indicates otherwise. Two specific mutations are identified herein in the promoter region of TPMT. The mutations affect a region that normally contains a series of six sequential repeats of the motif GCC, resulting in either the loss or gain of one repeat to give GCC₍₅₎ or GCC₍₇₎ respectively. It is anticipated that any mutation in this region, resulting in the loss or gain of one or more GCC repeats, will be associated with the UM phenotype and risk of thiopurine intolerance or resistance.

In a first aspect, the present invention provides a method for screening individuals for the presence or absence of one or more mutations associated with the UM phenotype and with the risk of thiopurine resistance or intolerance, which method includes the step of determining the genotypic state of the individual with respect to the TPMT gene promoter.

The mutation may comprise one or more additional GCC repeat sequences, or a loss of one or more GCC repeat sequences. Preferably, the mutation comprises the addition of a single GCC repeat GCC₍₇₎ (SEQ ID NO: 4), or a loss of a single GCC repeat GCC₍₅₎ (SEQ ID NO:3).

The genotypic state may be determined with respect to DNA obtained from said individual, by direct or indirect methods.

Preferably a DNA sample is obtained from an individual and the genotypic state of the TPMT promoter is assessed for the presence of at least one difference in the GCC repeat region of the promoter from the nucleotide sequence encoding TPMT (SEQ ID NO: 1), either by direct or indirect methods.

More preferably the genotypic state is determined by the presence of a mutation in the GCC repeat region of the TPMT promoter. This may be determined as part of a personal genome sequence in which trait data is determined from the genome sequence itself by direct comparison with known traits, or the mutation may be determined specifically. The mutation may consist of the loss of one or more GCC repeat sequences or the gain of one or more GCC repeat sequences. Preferably the mutation consists of a loss of a single GCC repeat or the gain of a single GCC repeat selected from SEQ ID NO 3 or 4 respectively, either by direct or indirect methods.

In another embodiment the invention provides a method of identifying an individual at risk of thiopurine resistance or intolerance, said method comprising:
obtaining a DNA sample from said individual and identifying a mutation in the GCC repeat region of the TPMT promoter, wherein the presence of said mutation is associated with a UM phenotype and a risk of thiopurine resistance or intolerance.

The mutation may consist of one or more additional GCC repeat sequences or a loss of one or more GCC repeat sequences.

Preferably the mutation consists of the addition of a single GCC repeat GCC₍₇₎ (SEQ ID NO:4), or a loss of a single GCC repeat GCC₍₅₎ (SEQ ID NO:3).

In still a further aspect, the present invention provides an isolated nucleic acid molecule suitable for use in detecting a mutation in the GCC repeat motif of the TPMT promoter. The mutation is preferably selected from the group consisting SEQ ID NO 3 or 4, and the nucleic acid molecule of the invention may consist of a nucleotide sequence having about at least 15 contiguous bases of SEQ ID NO 1 or a complementary sequence thereof.

In one embodiment, the nucleic acid molecule consists of a probe having a sequence which binds to the nucleotide sequence which contains at least one mutation of the invention.

In another embodiment, the nucleic acid molecule consists of a primer having a sequence which binds to the TPMT promoter either upstream or downstream of a mutation of the invention. The primer in a preferred embodiment binds to the TPMT promoter sequence upstream or downstream of the GCC sequential repeat motif and up to one base from said GCC repeat motif.

The mutation may comprise the loss or gain of one or more GCC repeat motifs. Preferably, the mutation comprises the loss or gain of a single GCC repeat motif to give GCC₍₅₎ or GCC₍₇₎ respectively.

In a still further aspect, the present invention provides an isolated nucleic acid molecule having the sequence of SEQ ID NO:1 and comprising a mutation in the GCC repeat motif. The mutation may comprise the loss or gain of one or more GCC repeat motifs. Preferably the mutation is selected from the group comprising SEQ ID NO 3 or 4, or a functional fragment, variant or antisense molecule thereof.

The nucleic acid molecule may alternatively comprise peptide nucleic acid (PNA). The nucleic acid may further comprise a detectable label, preferably a fluorescent label or a radioisotopic label. Alternatively, the genomic DNA sample may be fluorescently or radioisotopically labeled.

In another aspect, the invention relates to purified peptides encoded by the polynucleotide molecules of the invention, as well as antibodies raised against these peptides.

In another aspect, the invention provides a use of the GCC mutation in the TMPT promoter identified herein, in identifying individuals having a UM phenotype and at risk of thiopurine resistance or intolerance based on assessment of a personal genome sequence of said individual.

The mutation may comprise the loss of one or more GCC repeat sequences, or the gain of one or more GCC repeat sequences. Preferably the mutation comprises the loss or gain of a single GCC repeat sequence and is selected from the group comprising SEQ ID NO 3 or 4.

In another aspect, the present invention provides a diagnostic kit for identifying individuals having a UM phenotype and at risk of thiopurine resistance or intolerance based on assessment of the genotypic state of the TPMT promoter.

In a preferred embodiment, the kit comprises a probe of the invention.

Alternatively, the kit comprises a primer that binds to the TPMT promoter or the anti-sense strand thereof up to a nucleotide positioned one base from the GCC sequential repeat motif. The primer may be upstream or downstream of said motif.

In a further aspect, the present invention provides a diagnostic kit for identifying individuals having a UM phenotype and being at risk of thiopurine resistance or intolerance comprising first and second primers which are complementary to nucleotide sequences of the TPMT promoter or the anti-sense strand thereof upstream and downstream, respectively, of a mutation in the GCC sequential repeat motif.

The mutation may comprise the loss of one or more GCC repeat sequences, or the gain of one or more GCC repeat sequences. Preferably the mutation comprises the loss or gain of a single GCC repeat sequence and is selected from the group comprising SEQ ID NO 3 or 4.

The invention will now be described with reference to the sequences and figures of the accompanying drawings in which:

### Sequences

SEQ ID NO: 1 is the genomic sequence for the TPMT promoter as generated by UCSC Genome Browser (http://www.genome.ucsc.edu, March 2006 Assembly);

SEQ ID NO: 2 is a partial genomic sequence from the TPMT promoter showing the non-mutated GCC sequential repeat motif (GCC₍₆₎);

SEQ ID NO:3 is a partial genomic sequence from the TPMT promoter showing a mutation in the GCC sequential repeat motif, wherein the mutation comprises a loss of one repeat to give GCC₍₅₎.

SEQ ID NO:4 is a partial genomic sequence from the TPMT promoter showing a mutation in the GCC sequential repeat motif, whereby the mutation comprises a gain of one repeat to give GCC₍₇₎.

### BRIEF DESCRIPTION OF THE FIGURES

FIG. 1 shows the tri-modal distribution of TPMT activity in the population;

FIG. 2 shows a schematic diagram of azathioprine metabolism;

FIG. 3a shows the genomic organisation of the TPMT promoter;

FIG. 3b shows the wild-type human TPMT promoter sequence aligned with the sequences of eight other mammalian species showing the degree of conservation of the GCC repeat motif;

FIG. 4 shows electropherograms of the wild-type TPMT promoter sequences and variant promoter sequences showing the mutations of the invention; and

FIG. 5 shows the activity of the wild-type and variant TPMT promoters in reporter gene assays.

### DETAILED DESCRIPTION OF THE INVENTION

### Definitions

The term “drug” as used herein refers to a chemical entity administered to a person in a medical context to treat or prevent or control a disease or condition.

The term “therapy” refers to a process which is intended to produce a beneficial change in the condition of an individual. A beneficial change can, for example, include one or more of: restoration of function, reduction of symptoms, limitation or retardation of progression of a disease, disorder, or condition or prevention, limitation or retardation of deterioration of an individual's condition, disease or disorder.

In the context of the present invention, “thiopurine therapy” involves the administration to an individual of a thiopurine drug. Non-limiting examples of thiopurine drugs are azathioprine (Imuran®, Azamun®, Thiprine®) and 6-mercaptopurine (Puri-Nethol®).

In this specification “thiopurine intolerance” means an adverse reaction, such as liver toxicity, in individuals undergoing thiopurine therapy.

“Thiopurine resistance” means a lack of a desired therapeutic outcome in individuals undergoing thiopurine therapy.

“Individual” means a human being.

“Mutation” in the present invention means a variant form of a gene with reference to the GCC sequential repeat sequence in the promoter sequence of the TPMT gene and which is associated with a UM phenotype.

“Primer” refers to a single-stranded nucleic acid molecule, also referred to as an oligonucleotide, which specifically hybridizes (binds) to a predetermined region of DNA of complementary sequence. Primers are key reagents in polymerase chain reactions (PCR), and in a variety of polymorphism detection methods. They provide specific initiation sites for the polymerase enzymes used in PCR and in many polymorphism detection methods.

“Genotyping” or “Genotypic state” refers to a range of methods, including those reviewed by Kwok (2001), which determine the nature of the nucleotide(s) at a polymorphism or mutated site in the DNA of an individual.

The term “comprising” as used in this specification and claims means “consisting at least in part of”; that is to say when interpreting statements in this specification and claims which include “comprising”, the features prefaced by this term in each statement all need to be present but other features can also be present. Related terms such as “comprise” and “comprised” are to be interpreted in similar manner.

### Description

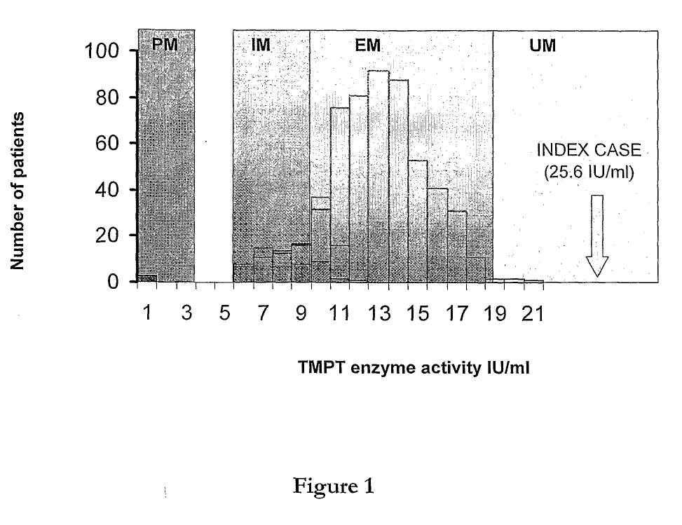

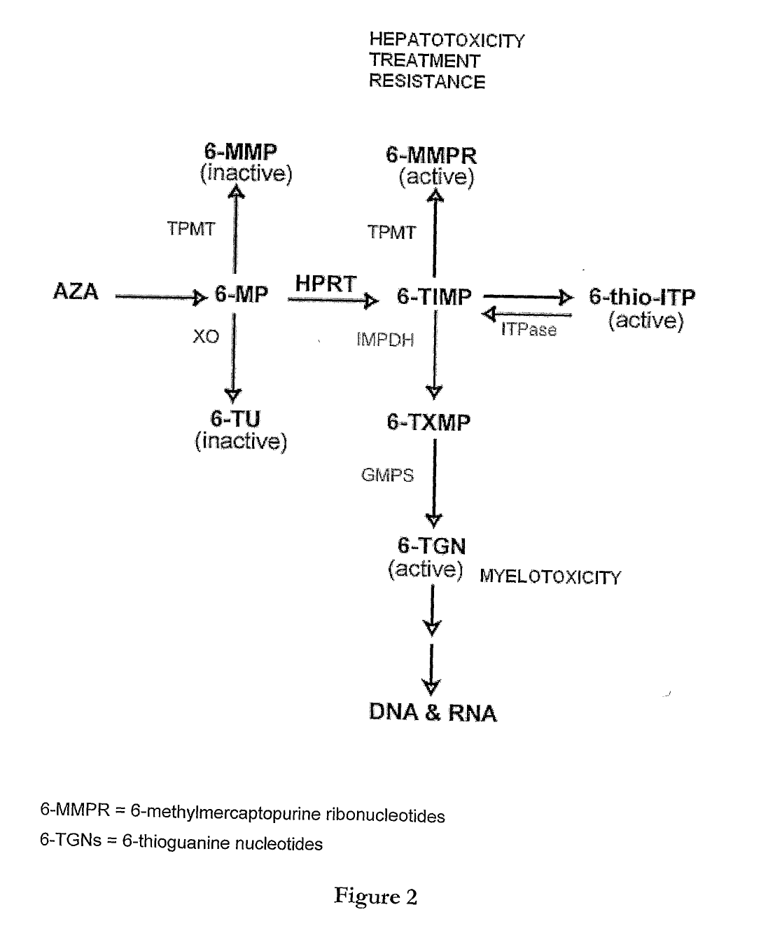

Thiopurine S-methyltransferase (TPMT; EC 2.1.1.67) is a cytosolic enzyme that catalyses the S-methylation of the thiopurine drugs azathioprine (AZA) and 6-mercaptopurine (6-MP). This enzyme is polymorphic with around 0.6% of Caucasians exhibiting complete deficiency (poor methylators—PM), 11% intermediate activity (intermediate methylators—IM) and 89% normal TPMT activity (extensive methylators—EM) in red blood cells (RBCs) (Wang et al, 2006). In addition 1-2% of Caucasians fall outside this tri-modal distribution and exhibit ultra-high TPMT activity (ultra-high methylators, UMs) (FIG. 1). As the thiopurine immunosuppressants have a relatively narrow therapeutic range, mutations in the TPMT gene play an important role in determining the ratio of two key active thiopurine metabolites, 6-methylmercaptopurine ribonucleotides (6-MMPR) and thioguanine nucleotides (6-TGN), and thereby the efficacy and toxicity of thiopurine therapy (see FIG. 2). Individuals who are PMs, and to a lesser extent IMs, are at a heightened risk of myelosuppression on standard doses of AZA and 6-MP due to elevated 6-TGN concentration. Conversely, individuals with ultra-high TPMT activity are more likely to experience treatment resistance (due to sub-therapeutic 6-TGN concentrations) and hepatotoxicity as a result of elevated 6-MMPR concentrations. The molecular basis of the majority of TPMT deficiency is now well-established and is one of the few examples where a pharmacogenetic phenomenon has been translated into a widely used diagnostic test to guide prescribing. In contract, the cause of significantly elevated enzyme activity is unknown, although a strong familial correlation suggests this phenomenon may have a genetic basis.

The present inventors have, for the first time, found mutations in the TPMT promoter trinucleotide (GCC) repeat region in patients exhibiting extremely high TPMT activity. Such high TPMT activity is associated with a risk of thiopurine resistance or intolerance.

Accordingly, in a first aspect, the present invention provides a method for screening an individual for the presence or absence of a mutation associated with extremely high levels of TPMT activity (the UM phenotype) and with the risk of thiopurine resistance or intolerance. The method includes at least the step of determining the genotypic state of the individual with respect to the TPMT promoter.

The genotypic state may be determined with respect to DNA obtained from said individual, by direct or indirect methods.

The GCC sequential repeat region of the TPMT promoter normally comprises six GCC repeats (SEQ ID NO:2).

Two mutations in the GCC sequential repeat region of the TPMT promoter have been identified and shown to be associated with the UM phenotype and with a risk of thiopurine resistance or intolerance in individuals undergoing such therapy. These mutations include a loss of a single GCC repeat sequence to give GCC(s) (SEQ ID NO:3), or a gain in a single GCC repeat sequence to give GCC₍₇₎ (SEQ ID NO:4).

However, it is anticipated that any mutation in this region, resulting in the loss or gain of one or more GCC repeats, will be associated with the UM phenotype and risk of thiopurine intolerance or resistance.

In some individuals, the presence of one or more polymorphisms or mutations has been shown to result in drug resistance, whilst other individuals may exhibit drug intolerance. There are some individuals who exhibit both thiopurine intolerance and resistance. By linking specific polymorphisms or mutations with therapeutic outcomes, a risk profile can be established to identify individuals at risk of thiopurine intolerance and/or resistance who have the same polymorphism or mutation.

Therefore, in another embodiment the invention provides a method of identifying an individual at risk of thiopurine resistance or intolerance, said method comprising:
obtaining a DNA sample from said individual and identifying a mutation in the GCC repeat region of the TPMT promoter, wherein the presence of said mutation is associated with a UM phenotype and with a risk of thiopurine resistance or intolerance.

The mutation may consist of a loss or gain of one or more GCC repeat sequences. Preferably the mutation consists of a loss of a single GCC repeat sequence (SEQ ID NO:3), or a gain of a single GCC repeat sequence (SEQ ID NO:4).

Determining such mutations enables records to be kept on the progress of individuals in thiopurine therapy. A mutation profile can therefore be established linking a probability of thiopurine intolerance or resistance with a particular mutation based on results from groups of individuals with identical or similar TPMT mutations.

Using mutation profiles, individuals can then be more accurately assessed as to whether thiopurine therapy is likely to be effective. Where a low probability of successful therapy is found, alternative treatments can be used including methotrexate, infliximab and other biological agents. Alternatively, individuals may proceed directly to surgery where necessary.

Profiles can also be used to determine appropriate therapeutic dosage and frequency ranges for thiopurine therapy by comparing successful therapies of various dosage and frequency ranges in individuals with similar or identical mutations.

Other risk factors can be combined into the analysis by a medical practitioner to support a prognosis of successful thiopurine treatment. These risk factors could be clinical or genetic and may include mutations in TPMT such as TPMT*2, TPMT*3A and TPMT*3C and others (Wang et al. 2006), guanosine 5′monophosphate synthetase (GMPS; EC 6.3.5.2) enzyme activity or genotype inosine triphosphate pyrophosphatase (ITPA) enzyme activity or genotype, and inosine 5′ monophosphate dehydrogenase (IMPDH; EC 1.1.1.205) enzyme active or genotype (of IMPDH1 and IMPDH2) (Roberts et al, 2006).

An individual's genotypic state is determined by comparing the TPMT promoter sequence, and specifically the GCC repeat sequence of the TPMT promoter of said individual to that of SEQ ID NO:1. The presence of at least one nucleotide difference in the individual's sequence from the nucleotide sequence of SEQ ID NO: 1 (determined by direct or indirect methods) is indicative of a polymorphism or mutation. Preferably, the presence of at least one GCC repeat motif difference is indicative of a polymorphism or mutation. This may comprise a gain in one or more GCC repeat motifs, or a loss in one or more GCC repeat motifs. A grouping into a majority or polymorphic mutation minority is enabled permitting different probabilities for therapeutic success to be determined.

In particular, it is contemplated that the novel mutations in the TPMT promoter GCC repeat sequence of the present invention may be used in personal genome sequencing to identify individuals at risk of thiopurine resistance or intolerance.

Personal genome sequencing, whilst currently in its infancy, will very soon become broadly available at modest cost. Personal genome sequencing is where individuals will have their own genome sequenced so that their genetic information may be used to identify risk profiles of disease, their physical and biological characteristics, and their personal ancestries. The genome sequence is compared to known mutations linked to various diseases or predisposition to diseases and biological characteristics. The present novel TPMT promoter mutations can be included in the personal genome databases for use in compiling disease risk profiles.

The TPMT promoter contains a variable number tandem repeat (VNTR), ranging from three to nine repeats (*V3 to *V9), located 43 bp upstream of the major transcription start site (Alves et al, 2000). Spire-Vayron de la Moureyre et al, 1999, conducted reporter gene assays and found that the *V7 allele significantly reduced luciferase activity and *V8 significantly increased luciferase activity relative to the three most common alleles (*V4, *V5, *V6). However, when 53 unrelated Caucasians were grouped according to their VNTR genotype, no statistical difference was observed between VNTR genotypes and mean TPMT activity in RBCs (Alves et al, 2000). Subsequent studies have been unable to establish a clear-cut relationship between repeat number and in vivo RBC TPMT activity. At best, it appears that this promoter VNTR only has a modest effect on the level of enzyme activity (Fessing et al, 1998). Therefore, to date, no molecular explanation for TPMT UM status has been found.

The present invention has found, for the first time, a mutation in another repeat sequence of the TPMT promoter, the GCC trinucleotide repeat sequence, which is directly linked to the UM phenotype in patients and with a risk of thiopurine resistance or intolerance.

One method for identifying an individual with a UM phenotype and at risk of thiopurine resistance or intolerance may comprise obtaining a DNA sample from said individual. The sample is then analysed to identify a mutation in the GCC trinucleotide repeat sequence of the TPMT promoter. Preferably the mutation comprises a loss or gain of one or more GCC repeat motifs. More preferably, the mutation is selected from the group consisting of SEQ ID NOs: 3 and 4 of the TPMT promoter. If the sample indicates the presence of said mutation, the individual is associated with a risk of thiopurine resistance or intolerance.

There are many experimental methods well known and available to art-skilled workers for determining the presence of additional mutations in the PMT promoter. These include, for example, methods based on PCR, methods based on denaturing high pressure liquid chromatography, DNA sequencing including personal genome sequencing, chemical or enzymatic analysis of mismatched DNA, and electrophoretic detection of mismatched DNA. Some specific examples of these methods are described in more detail below.

The application of these methods to TPMT may provide identification of additional mutations that can affect inter-individual probabilities of thiopurine drug intolerance or resistance. One skilled in the art will recognize that many such general methods have been described and can be utilized, as for example, reviewed by Syvanen and Taylor (2004).

The primers of the invention may be used in determining the presence or absence of a mutation in the GCC trinucleotide repeat sequence of the TPMT promoter in an individual. The presence or absence of specific mutations can be determined by a variety of methods, as recognized by those skilled in the art. For example, by chain termination methods, ligation methods, hybridization methods or by mass spectrometric methods.

A preferred embodiment of the method involves contacting an isolated TPMT promoter nucleic acid sequence of an individual with a nucleic acid probe which specifically identifies the presence or absence of a mutation in the GCC trinucleotide repeat sequence of the promoter. For example, a nucleic acid probe can be used which specifically binds, e.g., hybridizes, to a nucleic acid sequence corresponding to a portion of the promoter which includes at least one mutation under selective binding conditions.

Therefore, in another aspect, the present invention is directed to an isolated nucleic acid molecule suitable for use in detecting a mutation in the GCC trinucleotide repeat sequence of the TPMT promoter sequence, said nucleic acid molecule consisting of a nucleotide sequence having about at least 15 contiguous bases of SEQ ID NO 1 or a complementary sequence thereof.

The mutation comprises a loss or a gain of one or more GCC repeat motifs. Preferably, the mutation is selected from a loss of a single GCC repeat to give GCC₍₅₎ (SEQ ID NO:3) or a gain in a single GCC repeat to give GCC₍₇₎ (SEQ ID NO:4).

In one embodiment, the nucleic acid molecule consists of a probe having a sequence which binds to the nucleotide sequence which contains at least one mutation of the invention.

In another embodiment, the nucleic acid molecule consists of a primer having a sequence which binds to the TPMT promoter either upstream or downstream of a mutation. The primer in a preferred embodiment binds to the TPMT promoter sequence upstream or downstream of a mutation and up to one base from said mutation.

In a still further aspect, the present invention provides an isolated nucleic acid molecule having the sequence of SEQ ID NO:1 and comprising one or more mutations in the GCC repeat region. Preferably the mutation comprises a loss or gain of one or more GCC repeat motifs. More preferably the mutation is selected from the group comprising SEQ ID NOs 3 or 4, or a functional fragment, variant or antisense molecule thereof.

The nucleic acid molecule may alternatively comprise peptide nucleic acid (PNA). The nucleic acid may further comprise a detectable label, such as a fluorescent label a radioisotopic labels. Alternatively, the genomic DNA sample may be fluorescently or radioisotopically labelled.

Detecting mutations in gene sequences, including detecting mutations in repeat nucleotide sequences, can be accomplished by methods known in the art. For example, standard techniques for genotyping for the presence of single nucleotide polymorphisms (SNPs) and/or SSR markers can be adapted to detect nucleotide repeat motifs, including fluorescence-based techniques (Chen et al., 1999), utilizing PCR, LCR, Nested PCR and other techniques for nucleic acid amplification. Specific methodologies available for SNP genotyping include, but are not limited to, TaqMan genotyping assays and SNPIex platforms (Applied Biosystems), mass spectrometry (e.g., MassARRAY system from Sequenom), minisequencing methods, real-time PCR, Bio-Plex system (BioRad), CEQ and SNPstream systems (Beckman), Molecular Inversion Probe array technology (e.g., Affymetrix GeneChip), BeadArray Technologies (e.g., Illumina GoldenGate and Infinium assays) and oligonucleotide ligation assay (OLA—Karim et at, 2000). By these or other methods available to the person skilled in the art, one or more mutations in the TPMT promoter, and specifically in the GCC trinucleotide repeat sequence of the promoter can be identified.

A number of methods are thus available for analysis of nucleotide repeat sequences. Assays for detection of repeat sequences fall into several categories, including, but not limited to direct sequencing assays including personal genome sequencing, fragment polymorphism assays, hybridization assays, computer based data analysis, methods based on denaturing high pressure liquid chromatography, and electrophoretic detection of mutated DNA. Protocols and commercially available kits or services for performing multiple variations of these assays are available. In some embodiments, assays are performed in combination or in hybrid (e.g., different reagents or technologies from several assays are combined to yield one assay). The following are non-limiting examples of assays are useful in the present invention.

### Direct Sequencing Assays

Mutations in the GCC repeat sequences of the TPMT promoter may be detected using a direct sequencing technique. In these assays, DNA samples, such as those derived from for example blood, saliva or mouth swab samples, are first isolated from a patient using any suitable method. In some embodiments, the region of interest is cloned into a suitable vector and amplified by growth in a host cell (e.g., a bacteria). In other embodiments, DNA in the GCC repeat region of the TPMT promoter is amplified using PCR. DNA is sequenced using any suitable method, including but not limited to manual sequencing using radioactive marker nucleotides, or automated sequencing. The results of the sequencing are displayed using any suitable method. The sequence is examined and the presence or absence of a mutation in the GCC repeat sequence is determined.

### PCR Assay

Mutations in the GCC repeat sequence of the TPMT promoter may also be detected using a PCR-based assay. The PCR assay may comprise the use of oligonucleotide primers to amplify a fragment containing the GCC repeat sequence. The presence of one or more additional repeats in the TPMT promoter results in the generation of a longer PCR product which can be detected by gel electrophoresis, and compared to the PCR products from individuals without a mutation in the GCC repeat sequence. The presence of one or more fewer repeats in the TPMT promoter results in the generation of a shorter PCR product which is likewise detectable.

### Fragment Length Polymorphism Assays

In addition, the presence of a mutation in the GCC repeat region of the TMPT promoter may be detected using a fragment length polymorphism assay. In a fragment length polymorphism assay, a unique DNA banding pattern based on cleaving the DNA at a series of positions is generated using an enzyme (e.g., a restriction endonuclease). DNA fragments from a sample containing a mutation in the GCC repeat sequence will have a different banding pattern, from samples that do not contain the mutated GCC repeat sequence.

### RFLP Assay

In addition, the presence of a mutation in the GCC repeat region of the TMPT promoter may be detected using a restriction fragment length polymorphism assay (RFLP). The region of interest is first isolated using PCR. The PCR products are then cleaved with restriction enzymes known to give a unique length fragment for a given mutated GCC repeat sequence. The restriction-enzyme digested PCR products may be separated by agarose gel electrophoresis and visualized by ethidium bromide staining. The length of the fragments is compared to molecular weight standards and fragments generated from test and control samples, to identify test samples containing a mutation in the GCC repeat sequence.

### CFLP Assay

Alternatively, a mutation in the GCC repeat region of the TMPT promoter may be detected using a CLEAVASE fragment length polymorphism assay (CFLP; Third Wave Technologies, Madison, Wis.; and U.S. Pat. No. 5,888,780).

### Hybridization Assays

Also contemplated is detection of a GCC repeat sequence mutation by hybridization assay. In a hybridization assay, the presence of absence of a mutated sequence is determined based on the ability of the DNA from the sample to hybridize to a complementary DNA molecule (e.g., a oligonucleotide probe). A variety of hybridization assays using a variety of technologies for hybridization and detection are available and would be understood by a skilled worker

### Mass Spectroscopy Assay

A MassARRAY system (Sequenom, San Diego, Calif.) may also be used to detect presence of amutation in the GCC repeat sequence of the TMPT promoter (See e.g., U.S. Pat. No. 6,043,031).

One of the simplest methods is PCR analysis. Based on the sequence of the mutant sequences provided herein, PCR primers can be constructed that are complementary to the region of the TPMT promoter sequence encompassing the mutation. A primer consists of a consecutive sequence of nucleotides complementary to any region in the promoter encompassing the position which is mutated in the mutant sequence. PCR primers complementary to a region in the wild-type sequence corresponding to the mutant PCR primers are also made to serve as controls in the diagnostic methods of the present invention. The size of these PCR primers range anywhere from five bases to hundreds of bases. However, the preferred size of a primer is in the range from 10 to 40 bases, most preferably from 15 to 32 bases. As the size of the primer decreases so does the specificity of the primer for the targeted region. Hence, even though a primer which is less than five bases long will bind to the targeted region, it also has an increased chance of binding to other regions of the template polynucleotide which are not in the targeted region and do not contain the mutated base. Conversely, a larger primer provides for greater specificity, however, it becomes quite cumbersome to make and manipulate a very large fragment. Nevertheless, when necessary, large fragments are employed in the method of the present invention.

To amplify the region of the genomic DNA of the individual patient who may be a carrier for a mutant allele, primers to one or both sides of the targeted position, i.e. the genomic DNA positions 688 to 705 of SEQ ID NO:1 (in the GCC of that region), are made and used in a PCR reaction, using known methods in the art (e.g. Massachusetts General Hospital & Harvard Medical School, Current Protocols In Molecular biology, Chapter 15 (Green Publishing Associates and Wiley-Interscience 1991)).

According to the method of the present invention, once an amplified fragment is obtained, it can be analysed in several ways to determine whether the patient has a mutant allele of the TPMT promoter. For example, the PCR fragments can be sized, for example by using a capillary electrophoresis system after incorporating fluorescent dNTPs in the PCR products. The size of the fragment would indicate whether a mutation was present or not. Alternatively, the amplified fragments can be sequenced and their sequence compared with the wild-type genomic DNA sequence of TPMT. If the amplified fragment contains one or more of the mutations described in the present invention, the patient is likely to have a UM phenotype and therefore be at risk of developing hematopoietic toxicity when treated with standard amounts of thiopurine drugs, e.g. mercaptopurine and azathioprine. Alternatively, a combination of PCR and RFLP analysis may be used to determine TPMT genotype of the individual.

In a preferred embodiment, as described above, fluorescent dNTPs are incorporated into PCR products and visualisation carried out using capillary electrophoresis using an automated DNA sequences/fragment analyser. In another preferred embodiment of the invention, two common primers are used, each of which is complementary to either side of the mutation site. Common primers are those which do not encompass the mutation sites, i.e. their sequences are common to both the wild-type and the mutant alleles. The primers are extended in opposite directions so that they amplify a relatively large fragment encompassing the site of mutation. The products of this reaction are subsequently resolved either by sizing on an electrophoretic apparatus (such as a capillary electrophoresis system), or subjected to DNA sequence analysis.

Genotyping approaches include methods that require allele specific hybridization of primers or probes; allele specific incorporation of nucleotides to primers bound close to or adjacent to the polymorphisms or mutations (often referred to as “single base extension”, or “minisequencing”); and allele-specific ligation (joining) of oligonucleotides (ligation chain reaction or ligation padlock probes); or by invasive structure specific enzymes (Invader assay).

Detection methods for use in genotyping can involve the use of systems based on electrophoretic separation in agarose or polyacrylamide gels, capillary electrophoresis columns containing proprietary polymers, differential fluorescent or radioactive signals, or detection of differential size or mass of reaction products. These methods variously employ primers that flank, or that lie adjacent to, or that include within their sequence or at their most 3′ position, any of the mutations of the invention.

Other methods for determining mutations are known to art-skilled workers as reviewed by Syvanen and Taylor (2004). One preferred example is the use of mass spectrometric determination of a nucleic acid sequence which is a portion of the TPMT promoter or a complementary sequence. Such mass spectrometric methods are known to those skilled in the art, and most of the genotyping assays referred to above could be adapted for the mass spectrometric detection of the TPMT mutations of the invention.

The above method aspects can be facilitated by the provision of kits.

In another aspect, the present invention provides a diagnostic kit for identifying individuals at risk of thiopurine resistance or intolerance based on assessment of the genotypic state of the TPMT promoter.

The kit may comprise a probe of the invention. Alternatively a primer of the invention may be employed. Said primer should bind to the TPMT promoter or the antisense strand thereof up to a nucleotide positioned one base from a mutation of the invention.

In a further aspect, the present invention provides a diagnostic kit for identifying individuals at risk of thiopurine resistance or intolerance comprising first and second primers which are complementary to nucleotide sequences of the TPMT gene upstream and downstream of said at least one mutation

Preferably the mutation is selected from the group comprising a loss or gain of at least one GCC repeat motif. More preferably, the mutation is selected from the group consisting of SEQ ID NOs: 3 or 4.

In another aspect, the present invention provides a kit for detecting an altered probability of thiopurine resistance or intolerance in an individual, which kit comprises a nucleotide of SEQ ID NO: 1.

In another aspect, the kit comprises a single primer which is substantially complementary to the TPMT promoter sequence and binds directly to the nucleotide sequence of at least one mutation of the invention.

In still a further aspect, the present invention provides a primer suitable for use in detecting a mutation in the GCC trinucleotide repeat sequences of the TPMT promoter sequence, said primer consisting of a nucleotide sequence having about at least 15 contiguous bases of SEQ ID NO:1. Preferably the mutation is selected from a loss or gain of at least one GCC repeat motif. More preferably the mutation is selected from the group consisting of SEQ ID NO: 3 or 4.

The kit is preferably adapted and configured to be suitable for identification of the presence or absence of one or more particular mutations, comprising a nucleic acid sequence corresponding to a portion of the TPMT promoter sequence.

The mutation or mutations to be detected in the GCC repeat region of the TPMT promoter are correlated with variability in a treatment response or tolerance to thiopurine therapy, and are indicative of a UM phenotype.

In preferred embodiments, the kit contains components (e.g., probes and/or primers) adapted or useful for detection of a mutation in the TPMT promoter indicative of a UM phenotype and a risk of thiopurine intolerance or resistance in a patient requiring such therapy, e.g. in a patient suffering from IBD.

It may also be desirable to provide a kit containing components adapted or useful to allow detection of a plurality of mutations indicative of a UM phenotype and an associated risk of thiopurine intolerance or resistance. Such additional components can, for example, independently include a buffer or buffers, e.g., amplification buffers and hybridization buffers, which may be in liquid or dry form, a DNA polymerase, e.g., a polymerase suitable for carrying out PCR (e.g., a thermostable DNA polymerase), a DNA ligase, e.g. a DNA ligase suitable for performing the ligase chain reaction, specialised probes (such as padlock probes), and deoxynucleoside triphosphates (dNTPs), dideoxynucleoside triphosphates (ddNTPs) or ribonucleotide triphosphates.

Preferably, the kit comprises several oligonucleotides that will hybridize specifically to the TPMT promoter. These oligonucleotides will enable specific amplification of TPMT nucleotides from human genomic DNA using PCR. Most preferably, these oligonucleotides will also enable specific genotyping of the TPMT gene by acting as primers, probes, or ligation substrates that enable differentiation of polymorphic alleles. Alternatively, these oligonucleotides may be suitable for use in emerging methods that do not depend on prior amplification of the starting DNA, such as Invader assays and ligation-based detection methods. Preferably the oligonucleotides or other kit components will include a detectable label, e.g., a fluorescent label, enzyme label, light scattering label, mass label, or other label.

The kit may also optionally contain instructions for use, which can include a listing of the mutations correlating with a TPMT UM phenotype and associated risk of thiopurine intolerance or resistance.

Preferably the kit components may include samples of “control” DNA, constituting genomic DNA from individuals with different alleles of each of the indicated mutations, or recombinant plasmids or PCR products containing sequences representing the mutations. This will enable quality control of the assay when applied in different laboratories.

The present invention is also directed to a diagnostic assay to determine the TPMT genotype of a person. For example, tissue containing DNA such as white blood cells, mucosal scrapings of the lining of the mouth, saliva, epithelial cells, pancreatic tissue, liver, et cetera, is obtained from an individual. Genomic DNA of the individual is isolated from this tissue by the known methods in the art, such as phenol/chloroform extraction. PCR primers of the invention are synthesized. The primers are preferably 10-40 bases long, most preferably 15-31 bases long. The primers are added, and using a standard PCR procedure, a TPMT fragment is amplified. Next, the amplified sequence is analysed by the various methods described above, which include length or mass analysis, sequencing, mutation-specific amplification, or a combination of such methods and a diagnosis made based on the results.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples only.

### Example 1

The RBC TPMT activity of a 50-year-old Caucasian male (Patient A), with indeterminate colitis, was found to be high at 25.6 units/ml RBC (referred to as “index case” in FIG. 1) as determined using a radiochemical assay (Weinshilboum et al, 1999). TPMT phenotyping was first offered as a clinical service in Christchurch (New Zealand) in 2003 (Sies et al, 2005), and since then over 2000 individuals have been tested. The normal range of TPMT activity, as determined by the phenotyping assay, is 9.3 to 17.6 units/ml RBC (FIG. 1). The highest activity previously recorded was 22.5 units/ml RBC (Sies et al, 2005). As Patient A was not receiving any medication known to induce TPMT activity, it is conceivable that his UM status was due to a mutation within the TPMT promoter. To test this hypothesis, the TPMT promoter was sequenced in this patient and nine other individuals with UM phenotypes (18.4-22.5 units/ml RBC).

Genomic DNA was extracted from 5 ml of Patient A's peripheral blood using a NaCl method (Lahiri et al, 1991). The promoter and 5′UTR of TPMT were amplified in a 1225 bp fragment. The PCR was performed in a total volume of 25 μl containing 200 μM of dNTPs, 0.5 μM of each primer, 1.5 mM MgCl2, 1 U of DNA Taq polymerase (Roche), 1× Q-solution (Qiagen) and 100 ng of gDNA. Thermal cycling conditions were: 95° C. @ 15 minutes, followed by 35 cycles of 94° C. for 1 minute, 62° C. for 30 seconds, 72° C. for 2 minutes; and a final extension of 72° C. for 4 minutes. Five microlitres of each PCR was checked on 1% LE Agarose. An additional 5 μl aliquot was purified with Exo-SAP-IT® (USB Corporation, Cleveland, Ohio, USA) as per the manufacturer's instructions and sequencing using BigDye® chemistry (Applied Biosystems, CA, USA) on an ABI3730 Genetic Analyzer.

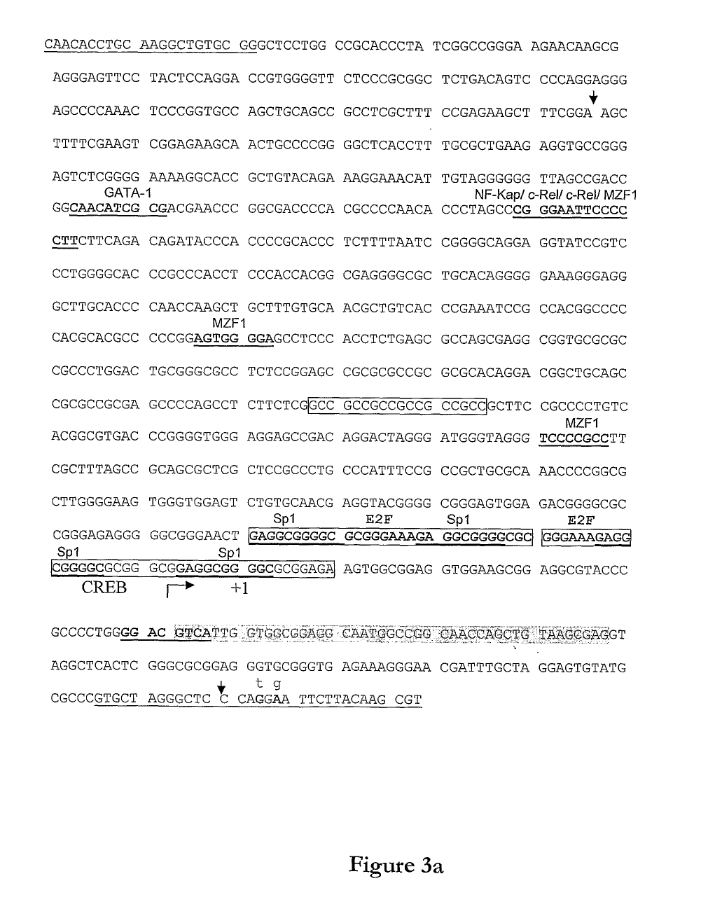

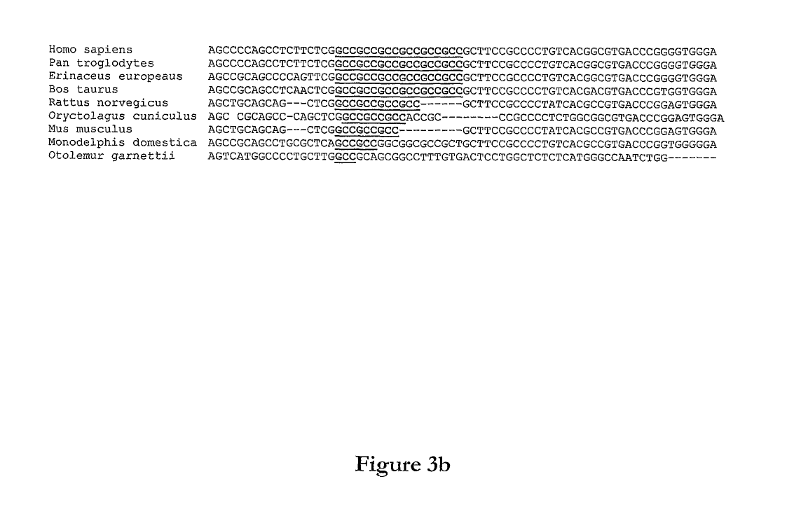

FIG. 3a shows the genomic PMT promoter sequence. The forward and reverse primer sequences are underlined and the bases altered in the reverse primer to create a NcoI recognition site are shown in bold. Vertical arrows indicate the cut sites of HindIII and NcoI, respectively. Transcription factor binding sites within the promoter were identified using TFSearch web-based software and sites found to have a score of >90.0 are shown in bold and underlined. The GCC trinucleotide repeat and the downstream variable number of tandem repeat (VNTR) element are boxed (the latter by dashed lines). Each repeat within the VNTR element consists of a 17-18 bp imperfect unit with a 14 bp core consensus sequence. The most common VNTR alleles consist of four or five repeats (VNTR*4 & VNTR*5)(Spire-Vayron de la Moureyre et al 1999). Patient A was homozygous for the VNTR*V4 allele. The major transcription start site (position+1) of TPMT is indicated by an arrow and exon 1 is shaded.

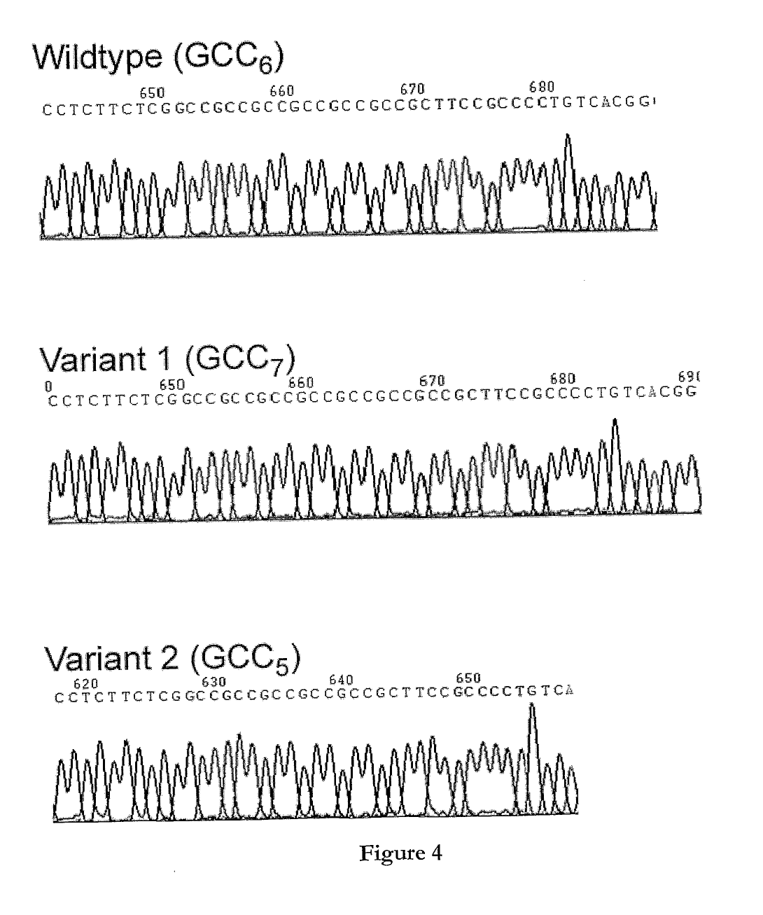

DNA sequencing revealed that Patient A was heterozygous for an additional GCC motif in a trinucleotide repeat that normally contains six repeats, located 324 bp upstream of the TPMT transcription start site (FIGS. 3a & 4). This trinucleotide repeat variant has not been reported in Ensembl (http://www.ensembl.org/index.html) or dbSNP (http://www.ncbi.nlm.nih.gov/entrez/query.fcgi?db=snp&cmd=search&term=). The promoters of an additional nine local UM patients (range: 18.4-22.5 units/ml RBC), 50 patients with normal TPMT activities (range: 12.5-16.5 units/ml RBC), and 200 Caucasion controls were screened to determine whether GCC₍₇₎ was a common polymorphism or a variant specifically associated with elevated TPMT activity. All 59 IBD patients were homozygous for GCC₍₆₎, whereas five of the 200 controls were heterozygous for the GCC₍₇₎ variant, suggesting that GCC₍₇₎ could be a rare mutation associated with the patient's extreme TPMT activity. An additional three patients with TPMT RBC activities comparable to Patient A (range: 55-65 nmol 6-MTG×g-1 Hb×h-1 normal range 26-50) were sourced from Guy's and St Thomas Hospital (London, United Kingdom). These patients exhibited TPMT activities within the 99^{th} percentile of enzyme distribution. All of them, however, were free of medical conditions (e.g. auto-immune haemolytic anemia) and medications (e.g. erythropoietin) that may result in a young RBC population and thus elevated TPMT activity. DNA sequencing revealed that one of these patients (Patient B; RBC activity 65 nmol 6-MTG×g-1 Hb×h-1) was heterozygous for a different variant, GCC₍₅₎, in the same GCC repeat element (FIG. 4). The other two UK patients were both homozygous for the wild-type GCC₍₆₎ allele (FIG. 4). All patients in this study were heterozygous or homozygous for the VNTR*V4 or VNTR*V5 allele. Furthermore, no additional sequence variations in TPMT, which could potentially account for elevated TPMT activity, were found in any of the UMs.

To further evaluate the association discovered between the promoter GCC₍₅₎ and GCC₍₇₎ alleles and TPMT UM activity, additional patients on thiopruine treatment were sourced from the Department of Clinical Genetics, St Mary's Hospital, Manchester (United Kingdom). DNA from thirteen patients with inflammatory bowel disease or rheumatoid arthritis were provided for analysis, all of whom had TPMT UM phenotype (activity levels of >50 nmol/g Hb/h). Of these 13 patients, three proved heterozygous for a GCC₍₇₎ mutation, further confirming the disproportionate representation of TPMT GCC trinucleotide mutations amongst patients with a UM phenotype.

The wild-type human TPMT promoter sequence was aligned with the sequences from 21 additional mammalian species to determine the degree to which the trinucleotide repeat element is conserved (FIG. 3(b)). Alignment was performed using Ensembl (http://www.ensembl.org/index.html). Sequences that demonstrated some evidence of the repeat element are displayed in decreasing order of homology to the human TPMT sequence. For each sequence, the trinucleotide element is shown in bold and underlined. Regions of zero homology are indicated by dashed lines.

Alignment of the human TPMT promoter sequence revealed that nine of the 21 species showed evidence of the trinucleotide repeat element (FIG. 3b). The (GCC)₆ allele was observed in chimpanzee (P. troglodytes), hedgehog (E. europeaus), and cow (B. taurus), whereas the other six species showed lower repeat number. None of the mammals examined had the (GCC)₅ or (GCC)₇ allele.

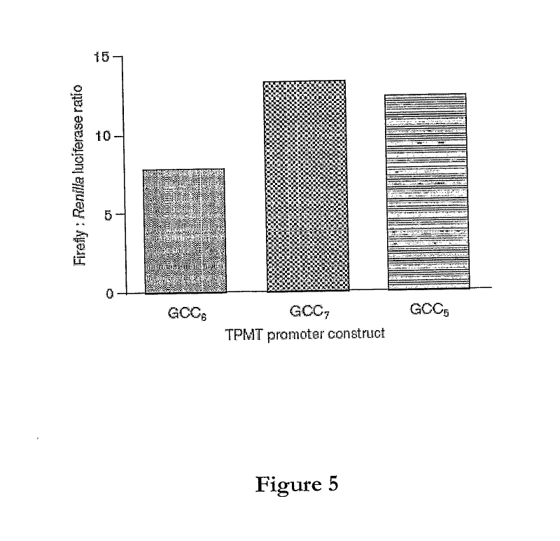

To assess the potential functional effect of the variation in the trinucleotide repeat region, the wild-type and variant TPMT promoters of Patient A and Patient B were amplified and cloned into the promoter-less pGL3-Basic vector (Promega Corporation, Madison, Wis., USA) using the restriction sites Hind III and NcoI. The construct was confirmed by direct DNA sequencing and purified using the EndoFree® Plasmid Kit (Qiagen). Twenty-four hours prior to transfection a 24-well tissue culture plate was seeded with COS-7 cells (5×10⁴ cells/well) to ensure 40-80% confluency. Each well contained 500 μl of Dulbecco's modified Eagle's medium (DMEM) supplemented with 10% fetal bovine serum and 100 ng/ml of penicillin & streptomycin. Pre- and post-transfected cells were incubated at 37° C. at 5% CO2. Transfections were performed using Effectene® Transfection Reagent (Qiagen) as per the manufacturer's instructions. To control for variation in transfection efficiency among replicates, the promoter construct was co-transfected with the Renilla vector, pRL-TK, (Promega). At 48 hours post-transfection, COS-7 cells were lysed in Passive Lysis Buffer (Promega), and firefly luciferase and Renilla luciferase activities were measured sequentially using the Dual-Luciferase® Reporter Assay System (Promega). A promoterless pGL3-basic vector was used as a negative control in each transfection experiment (mean luciferase activity of control: 0.13±0.07). Differences in expression were assessed using the analysis of variance and Tukey's multiple comparison test and were considered to be statistically significant if P<0.05. To normalize for transfection efficiency, the promoter activity of the construct was expressed as the ratio of firefly luciferase activity to Renilla luciferase activity. The construct was tested in triplicate across three separate transfections. The mean normalized firefly luciferase activities of the constructs were 13.2±0.10 for GCC₍₇₎, 12.3±0.12 for GCC₍₅₎ and 8.0±0.26 for the wild-type GCC₍₆₎ across transfections. The difference in basal transcription activity between variant and wild-type constructs was highly significant (Tukey's multiple Comparison Test, p=<0.001). Furthermore, the expression difference observed between GCC₍₅₎ and GCC₍₇₎ was also significant (FIG. 5).

The molecular mechanism by which trinucleotide repeat variation might increase TPMT expression is unclear. A search for transcription factor binding sites using the web-based programmes TFSearch (http://www.cbrc.jp/research/db/TFSEARCH.html) and MatInspector (http://www.genomatix.de/) found that the two trinucleotide repeat polymorphisms (GCC₍₅₎ and GCC₍₇₎) did not create or abolish a transcription factor binding site (FIG. 3a). Without being bound by theory, although the trinucleotide repeat variation does not appear to alter a known transcription factor binding site, it is possible that the change in spacing between adjacent transcription factor binding sites, may have led to enhanced transcription. This phenomenon is not without precedence. A study of protein C gene transcription demonstrated that an engineered 3 bp insertion at position −21 of the promoter increased transcription activity 2.5 fold compared to the wildtype promoter in a CAT reporter assay (Spek et al, 1999). Similarly, Saxena et al 2006, reported novel 6-18 bp polymorphic insertions 190 bp upstream of the major transcription start site of the MCL-1 (myeloid cell leukemia-1) gene. Luciferase reporter assays in four cell lines demonstrated these insertions caused a 1.8 to 2.5 fold increase in MCL-1 activity. As in the case of the present variants, neither the engineered 3 bp insertion nor the MCL-1 polymorphisms created or abolished transcription factor binding sites. Spek et al, 1999, speculated that the increase in transcription observed with the engineered insertions may result from release of steric hindrance between adjacent transcription factors which in turn permitted a higher occupancy of binding sites and greater gene expression.

To date, 22 allelic variants (TPMT*2 to TPMT*23) associated with reduced TPMT enzyme activity have been identified (Wang et al, 2006, Lindquist et al 2007, Schaeffeler et al 2006). In contrast, no molecular explanation has been found for UM activity.

The present invention has identified and characterised two novel mutations in a trinucleotide repeat region, which result in increased in vitro basal expression of the TPMT promoter. These data further suggest that deviation of the number of repeat units in this promoter trinucleotide repeat region from the wild-type of six, may explain a proportion of the extreme TPMT UMs that are identified during routine phenotyping. To the inventors knowledge, no one has previously documented variation in this repeat element. In contrast the TPMT VNTR located >200 bp downstream of this trinucleotide repeat has received considerable attention (Spire-Vayron de la Moureyre et al, 1999; Krynetski et al 1997; Alves et al, 2000), and despite extensive in vitro studies (Spire-Vayron de la Moureyre et al, 1999; Alves et al 2001; Yan et al 2000), it appears these alleles only have a “modulatory” effect on TPMT expression. While the relevance of the TPMT promoter VNTR to thiopurine pharmacogenetics is unclear, the mutations discovered herein appear to have a clear effect on expression in vitro.

As these patients are at risk of thiopurine intolerance or resistance, and the associated significant side effects such as hepatoxicity, identification of such patients will be highly beneficial.

It is not the intention to limit the scope of the invention to the abovementioned examples only. As would be appreciated by a skilled person in the art, many variations are possible without departing from the scope of the invention as set out in the accompanying claims.

### REFERENCES

Kwok, P. Y. (2001). Methods for genotyping single nucleotide polymorphisms. Annu Rev Genomics Hum Genet 2, 235-258.
Syvanen, A. C., and Taylor, G. R. (2004). Approaches for analyzing human mutations and nucleotide sequence variation: a report from the Seventh International Mutation Detection meeting, 2003. Hum Mutat 23, 401-405.
Wang L, Weinshilboum R. Thiopurine S-methyltransferase pharmacogenetics: insights, challenges and future directions. Oncogene 2006; 25:1629-38.
Weinshilboum R M, Raymond F A, Pazmino P A. Human erythrocyte thiopurine methyltransferase: radiochemical microassay and biochemical properties. Clin Chim Acta 1978; 85:323-33.
Lahiri D K, Nurnberger J I, Jr. A rapid non-enzymatic method for the preparation of HMW DNA from blood for RFLP studies. Nucleic Acids Res 1991; 19:5444.
Sies C, Florkowski C, George P, Gearry R, Barclay M, Harraway J, Pike L, Walmsley T. Measurement of thiopurine methyl transferase activity guides dose-initiation and prevents toxicity from azathioprine. N Z Med J 2005; 118:U1324.
Roberts R L, Gearry R B, Barclay M L, Kennedy M A. IMPDH1 promoter mutations in a patient exhibiting azathioprine resistance. Pharmacogenomics J. 2007. 7(5) p312-7.
Fessing, M. Y., et al., Molecular cloning and functional characterization of the cDNA encoding the murine thiopurine S-methyltransferase (TPMT). FEBS Lett, 1998. 424(3): p. 143-5.
Spire-Vayron de la Moureyre, C., et al., Characterization of a variable number tandem repeat region in the thiopurine S-methyltransferase gene promoter. Pharmacogenetics, 1999. 9(2): p. 189-98.
Alves, S., et al., Characterization of three new VNTR alleles in the promoter region of the PMT gene. Hum Mutat, 2000. 15(1): p. 121.
Karim et al., 2000), Animal Genetics 31: 396-399)
Spek C A, Bertina R M, Reitsma P H. Unique distance- and DNA-turn-dependent interactions in the human protein C gene promoter confer submaximal transcriptional activity. Biochem J 1999; 340 (Pt 2):513-8.
Saxena A, Moshynska O V, Moshynskyy I D, Neuls E D, Qureshi T, Bosch M, et al. Short nucleotide polymorphic insertions in the MCL-1 promoter affect gene expression. Cancer Lett 2006.
Ktynetski E Y, Fessing M Y, Yates C R, Sun D, Schuetz J D, Evans W E. Promoter and intronic sequences of the human thiopurine S-methyltransferase (TPMT) gene isolated from a human PAC1 genomic library. Pharm Res 1997; 14:1672-8.
Alves S, Amorim A, Ferreira F, Prata M J Influence of the variable number of tandem repeats located in the promoter region of the thiopurine methyltransferase gene on enzymatic activity. Clin Pharmacol Ther 2001; 70:165-74.
Yan L, Zhang S, Eiff B, Szumlanski C L, Powers M, O'Brien J F, et al. Thiopurine methyltransferase polymorphic tandem repeat: genotype-phenotype correlation analysis. Clin Pharmacol Ther 2000; 68:210-9.
Lindquist M, Skoglund K, Karlgren A, Soderkvist P, Peterson C, Kidhall I, et al. Explaining PMT genotype/phenotype discrepancy by haplogyping of TPMT*3A and identification of a novel sequence variant, TPMT*23. Pharmacogenet Genomics 2007; 17:891-895.
Schaeffeler E, Eichelbaum M, Reinisch W, Zanger U M, Schwab M. Three novel thiopurine S-methyltransferase allelic variants (TPMT*20, *21, *22)-association with decreased enzyme function. Hum Mutat 2006; 27:976.
Chen, X. et al., Genome Res. 9(5): 492-98 (1999)

1. A method for screening individuals for the presence or absence of one or more mutations associated with the UM phenotype and with the risk of thiopurine resistance or intolerance, which method includes the step of determining the genotypic state of the individual with respect to the TPMT gene promoter.

2. A method as claimed in claim 1 wherein the genotypic state is determined with respect to DNA obtained from said individual, by direct or indirect methods.

3. A method as claimed in 2, wherein a DNA sample is obtained from an individual and the genotypic state of the TPMT promoter is assessed for the presence of at least one difference in the GCC repeat region of the promoter from the nucleotide sequence encoding TPMT (SEQ ID NO: 1), either by direct or indirect methods.

4. A method as claimed in claim 3, wherein the genotypic state is determined by the presence of a mutation in the GCC repeat region of the TPMT promoter.

5. A method as claimed in claim 4, wherein the mutation consists of the loss of one or more GCC repeat sequences or the gain of one or more GCC repeat sequences.

6. A method as claimed in claim 4, wherein the mutation consists of a loss of a single GCC repeat or the gain of a single GCC repeat selected from SEQ ID NO: 3 or 4 respectively.

7. A method as claimed in claim 1, wherein the genotype state of said individual is determined by personal genome sequencing.

8. A method of identifying an individual at risk of thiopurine resistance or intolerance, said method comprising: obtaining a DNA sample from said individual and identifying a mutation in the GCC repeat region of the TPMT promoter, wherein the presence of said mutation is associated with a UM phenotype and a risk of thiopurine resistance or intolerance.

9. A method as claimed in claim 8, wherein the mutation consists of one or more additional GCC repeat sequences or a loss of one or more GCC repeat sequences.

10. A method as claimed in claim 9, wherein the mutation consists of the addition of a single GCC repeat GCC₍₇₎ (SEQ ID NO:4), or a loss of a single GCC repeat GCC₍₅₎ (SEQ ID NO:3).

11. A method as claimed in claim 8, wherein the method comprises personal genome sequencing.

12. A use of a sequence comprising a GCC mutation in the TMPT promoter as defined in claim 3, to identify an individual having a UM phenotype and at risk of thiopurine resistance or intolerance, based on a personal genome sequence of said individual.

13. A use as claimed in claim 12, wherein the mutation consists of one or more additional GCC repeat sequences or a loss of one or more GCC repeat sequences.

14. A use as claimed in claim 12, wherein the mutation consists of the addition of a single GCC repeat GCC₍₇₎ (SEQ ID NO:4), or a loss of a single GCC repeat GCC₍₅₎ (SEQ ID NO:3).

15. An isolated nucleic acid molecule suitable for use in detecting a mutation in the GCC repeat motif of the TPMT promoter.

16. An isolated nucleic acid as claimed in claim 15, wherein the mutation is selected from the group consisting SEQ ID NO 3 or 4, and the nucleic acid molecule consists of a nucleotide sequence having about at least 15 contiguous bases of SEQ ID NO 1 or a complementary sequence thereof.

17. A nucleic acid molecule as claimed in claim 15, consisting of a probe having a sequence which binds to the nucleotide sequence which contains at least one mutation of the invention.

18. A nucleic acid molecule as claimed in claim 15, consisting of a primer having a sequence which binds to the TPMT promoter either upstream or downstream of a mutation defined in claim 5.

19. A nucleic acid as claimed in claim 18, wherein the primer binds to the TPMT promoter sequence upstream or downstream of the GCC sequential repeat motif and up to one base from said GCC repeat motif.

20. A nucleic acid molecule as claimed in claim 18, wherein the mutation comprises the loss or gain of one or more GCC repeat motifs.

21. A nucleic acid molecule as claimed in claim 20, wherein the mutation comprises the loss or gain of a single GCC repeat motif, to give GCC₍₅₎ or GCC₍₇₎ respectively.

22. An isolated nucleic acid molecule having the sequence of SEQ ID NO:1 and comprising a mutation in the GCC repeat motif.

23. A nucleic acid molecule as claimed in claim 22, wherein the mutation comprises the loss or gain of one or more GCC repeat motifs.

24. A nucleic acid molecule as claimed in claim 23, wherein the mutation is selected from the group comprising SEQ ID NO 3 or 4, or a functional fragment, variant or antisense molecule thereof.

25. A diagnostic kit for identifying individuals having a UM phenotype and at risk of thiopurine resistance or intolerance based on assessment of the genotypic state of the TPMT promoter.

26. A kit as claimed in claim 25, comprising a probe suitable for use in detecting a mutation in the GCC repeat motif of the TPMT promoter, said probe having a sequence which binds to the nucleotide sequence which contains at least one mutation of the invention.

27. A kit as claimed in claim 25, comprising a primer that binds to the TPMT promoter or the anti-sense strand thereof up to a nucleotide positioned one base from the GCC sequential repeat motif.

28. A kit as claimed in claim 27, wherein the primer is upstream or downstream of said motif.

29. A diagnostic kit for identifying individuals having a UM phenotype and being at risk of thiopurine resistance or intolerance comprising first and second primers which are complementary to nucleotide sequences of the TPMT promoter or the anti-sense strand thereof upstream and downstream, respectively, of a mutation in the GCC sequential repeat motif.

30. A diagnostic kit as claimed in claim 29, wherein the mutation comprises the loss of one or more GCC repeat sequences, or the gain of one or more GCC repeat sequences.

31. A kit as claimed in claim 30, wherein the mutation comprises the loss or gain of a single GCC repeat sequence and is selected from the group comprising SEQ ID NO 3 or 4 respectively.
